(19) United States
(12) Patent Application Publication
(10) Pub. No.: US 2024/0263991 A1
(43) Pub. Date: Aug. 8, 2024

(54) PROCESSING APPARATUS, PROCESSING METHOD, PROCESSING PROGRAM, AND BODY WEIGHT DISPLAY SYSTEM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Shotaro YASUDA, Taito-ku (JP)

(73) Assignee: TOYOTA JIDOSHA KABUSHIKI KAISHA, Toyota-shi (JP)

(21) Appl. No.: 18/435,194

(22) Filed: Feb. 7, 2024

(30) Foreign Application Priority Data
Feb. 8, 2023 (JP) 2023-017286

(51) Int. Cl. G01G 19/414; A63B 24/00; G01G 19/44

(52) U.S. Cl. CPC G01G 19/4146; A63B 24/0062; A63B 2024/0065; G01G 19/44

(57) ABSTRACT

A processing apparatus capable of preventing the motivation to lose weight from being reduced is provided. A processing apparatus according to one aspect of the present disclosure includes: a personal data acquisition unit configured to acquire personal data including body weight data of a dieter; a life data acquisition unit configured to acquire life data of the dieter; and a calculation unit configured to calculate a corrected body weight based on a correction term generated from the personal data and the life data and a measured body weight of the dieter.

### CROSS REFERENCE TO RELATED APPLICATIONS

This application is based upon and claims the benefit of priority from Japanese patent application No. 2023-17286, filed on Feb. 8, 2023, the disclosure of which is incorporated herein in its entirety by reference.

### BACKGROUND

The present disclosure relates to a processing apparatus, a processing method, a processing program, and a body weight display system.

For example, an other-self health/body shape forecasting system disclosed in Japanese Unexamined Patent Application Publication No. 2007-310632 simulates their own body shapes and health states after a certain period of time based on medical examination data, dietary data, and the like, displays the current other-self and the other-self after the elapse of the certain period of time, and displays a method for coping with the change in the body shape. According to this system, for example, it is possible to imagine the body shape of a person (hereinafter also referred to as a dieter) who is trying to lose weight (i.e., is on a diet) after the elapse of a certain period of time.

### SUMMARY

The applicant has found the following problem. In general, a dieter may gain weight in a weight loss plateau. Therefore, in the other-self health/body shape forecasting system disclosed in Japanese Unexamined Patent Application Publication No. 2007-310632, the body shape of a dieter on a diet after the elapse of a certain period of time is displayed as a body shape larger than the current body shape, and thus the motivation of the dieter to lose weight may be reduced.

The present disclosure has been made in order to solve the above-described problem and provides a processing apparatus, a processing method, a processing program, and a body weight display system that are capable of preventing the motivation to lose weight from being reduced.

A processing apparatus according to one aspect of the present disclosure includes:
a personal data acquisition unit configured to acquire personal data including body weight data of a dieter;
a life data acquisition unit configured to acquire life data of the dieter; and
a calculation unit configured to calculate a corrected body weight based on a correction term generated from the personal data and the life data and a measured body weight of the dieter.

In the above-described processing apparatus, it is preferable that the calculation unit estimate a weight loss plateau of the dieter based on a difference between a theoretical body weight calculated from the personal data and the life data and the measured body weight of the dieter, and calculate the corrected body weight in the weight loss plateau.

The above-described processing apparatus preferably includes a control unit configured to control a body weight scale so that it displays the corrected body weight.

In the above-described processing apparatus, it is preferable that the life data acquisition unit acquire exercise history data or meal history data of the dieter as the life data.

A body weight display system according to one aspect of the present disclosure includes:
the above-described processing apparatus; and
a body weight scale configured to display the corrected body weight.

The above-described body weight display system preferably includes a detection unit configured to detect a meal consumed by the dieter.

In the above-described body weight display system, the body weight scale preferably includes a selection unit configured to select whether to display the measured body weight or the corrected body weight of the dieter.

A processing method according to one aspect of the present disclosure includes:
acquiring personal data including body weight data of a dieter;
acquiring life data of the dieter; and
calculating a corrected body weight based on a correction term generated from the personal data and the life data and a measured body weight of the dieter.

A processing program according to one aspect of the present disclosure causes a computer to:
acquire personal data including body weight data of a dieter;
acquire life data of the dieter; and
calculate a corrected body weight based on a correction term generated from the personal data and the life data and a measured body weight of the dieter.

According to the present disclosure, it is possible to provide a processing apparatus, a processing method, a processing program, and a body weight display system that are capable of preventing the motivation to lose weight from being reduced.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration of a control system of a body weight display system according to a first embodiment;

FIG. 2 is a diagram showing a relation between each of a measured body weight, a corrected body weight, and a theoretical body weight of a person on a diet and time;

FIG. 3 is a flowchart showing a flow of processes performed by a processing apparatus according to the first embodiment; and

FIG. 4 is a block diagram showing a configuration of a control system of a body weight display system according to a second embodiment.

### DESCRIPTION OF EMBODIMENTS

Specific embodiments to which the present disclosure is applied will be described hereinafter in detail with reference to the drawings. However, the present disclosure is not limited to the following embodiments. Further, the following descriptions and the drawings are simplified as appropriate.

### First Embodiment

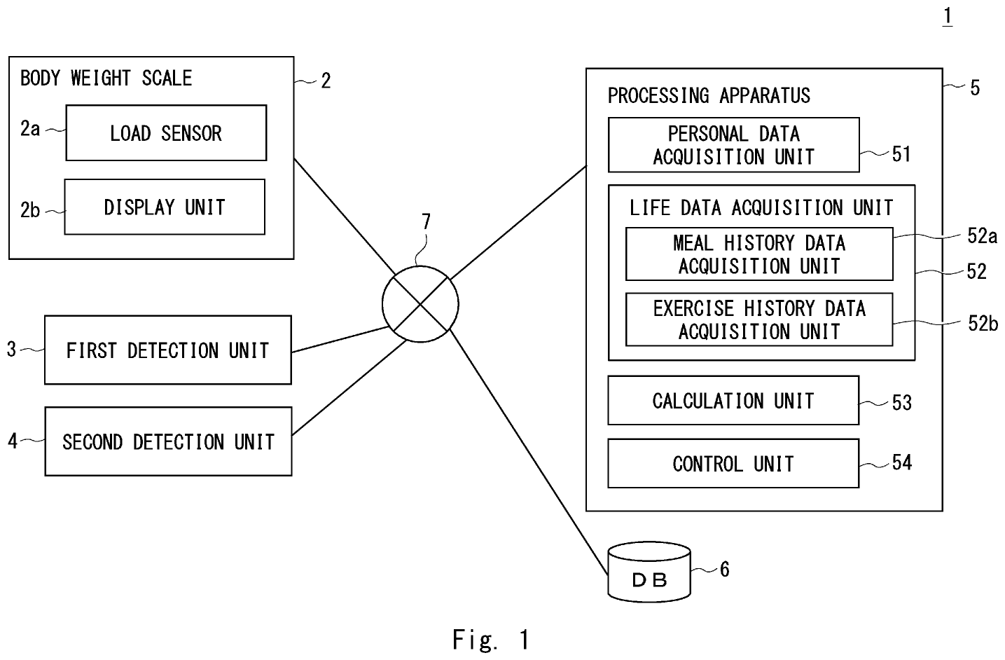

FIG. 1 is a block diagram showing a configuration of a control system of a body weight display system according to this embodiment. A body weight display system 1 according to this embodiment is suitably used to display the body weight of a dieter on a diet. As shown in FIG. 1, the body weight display system 1 includes a body weight scale 2, a first detection unit 3, a second detection unit 4, a processing apparatus 5, and a database (DB) 6.

The body weight scale 2, the first detection unit 3, the second detection unit 4, the processing apparatus 5, and the database 6 are connected to each other through a network 7. Note that the network 7 is a wired or a wireless communication line, for example, the Internet.

For example, a configuration of the body weight scale 2 is substantially equal to that of a general body weight scale, and the body weight scale 2 is configured to measure the body weight of a person to be measured (i.e., a dieter) standing on the body weight scale 2 by a load sensor 2a and display it on a display unit 2b. The body weight scale 2 transmits body weight data of the dieter to the DB 6.

The first detection unit 3 includes a sensor for detecting meals of a dieter. The first detection unit 3 includes an imaging sensor installed in, for example, a dining room at home, and acquires, as meal data, images obtained by imaging meals consumed by the dieter. Note that the meals are not limited to breakfast, lunch, and dinner, but also includes snacks between meals, midnight snacks, and the like. The first detection unit 3 associates meal data with meal time data and transmits it to the DB 6.

The second detection unit 4 includes a sensor for detecting exercise of a dieter. The second detection unit 4 includes, for example, a motion sensor which can be attached to a dieter, and acquires exercise data of the dieter. However, the second detection unit 4 may include a sensor capable of detecting activities of a dieter, and may include, for example, an imaging sensor.

Note that exercises are not only walking, jogging, and riding a bicycle, but also activities that burn calories as a whole, such as moving somewhere. The second detection unit 4 associates exercise data with exercise time data and transmits it to the DB 6.

As shown in FIG. 1, the processing apparatus 5 includes a personal data acquisition unit 51, a life data acquisition unit 52, a calculation unit 53, and a control unit 54. The processing apparatus 5 can be implemented by one or a plurality of computers etc. The processing apparatus 5 is typically an information processing apparatus such as a server apparatus.

Specifically, the processing apparatus 5 includes a memory that stores a program and the like, and a processor that executes the program. The processing apparatus 5 may include a plurality of processors for performing distributed processing.

The personal data acquisition unit 51 acquires personal data regarding a dieter. The personal data is data indicating the weight, the height, the age, the gender, and the like of the dieter. In this case, the personal data may include data indicating the body fat percentage, the body fat amount, or the muscle mass of a user. The personal data acquisition unit 51 can acquire the personal data from the DB 6.

The life data acquisition unit 52 includes a meal history data acquisition unit 52a and an exercise history data acquisition unit 52b. The meal history data acquisition unit 52a acquires meal history data of a dieter, which data is an example of life data of the dieter.

Specifically, the meal history data acquisition unit 52a estimates a meal menu based on meal data associated with meal time data acquired from the DB 6, and further estimates food included in the meal menu and the quantity thereof. The meal history data acquisition unit 52a then calculates the calorie intake of the meal menu based on the food included in the meal menu and the quantity thereof.

Note that calorie intake can be calculated using a method in which calorie intake and nutrients are calculated by performing an image analysis of image data obtained by imaging general meals. Thus, the meal history data acquisition unit 52a can acquire meal history data in which a meal menu, meal time, and calorie intake are associated with each other. The meal history data acquisition unit 52a transmits meal history data to the DB 6.

The exercise history data acquisition unit 52b acquires exercise history data of a dieter, which data is an example of life data of the dieter. Specifically, the exercise history data acquisition unit 52b estimates an exercise content and the amount of exercise based on exercise data associated with exercise time data acquired from the DB 6.

Note that the exercise content and the amount of exercise can be estimated using a method similar to that performed by a wearable terminal such as a general smartwatch. By this configuration, the exercise history data acquisition unit 52b can acquire exercise history data in which the exercise content, the exercise time, and the amount of exercise are associated with each other. At this time, the exercise history data may include, for example, data such as a heart rate and a body temperature. The exercise history data acquisition unit 52b transmits the exercise history data to the DB 6.

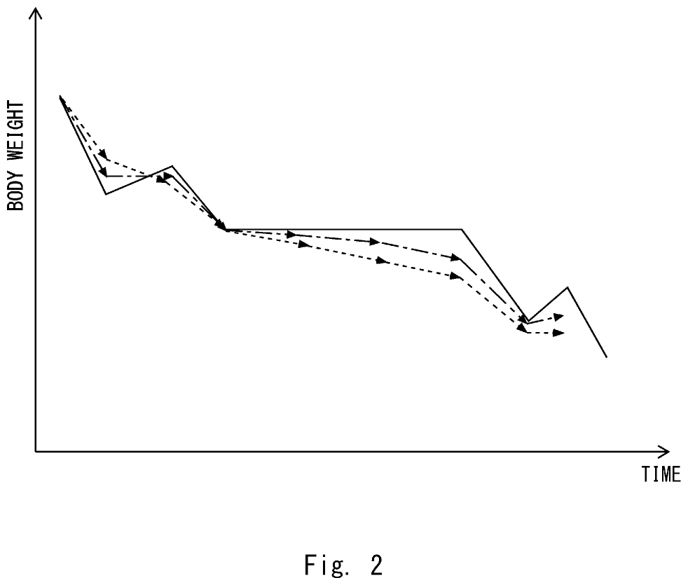

The calculation unit 53 calculates a corrected weight based on a correction term generated from the personal data and the life data and a measured body weight of a dieter. FIG. 2 is a diagram showing a relation between each of a measured body weight, a corrected body weight, and a theoretical body weight of a person on a diet and time. Note that, in FIG. 2, a solid line indicates the measured body weight, an alternate long and short dashed line indicates the corrected body weight, and a broken line indicates the theoretical body weight.

The corrected body weight can be calculated by the following <Expression 1>.

corrected body weight=current measured body weight−correction term <Expression 1>

In this Expression, the current measured body weight is the body weight of the dieter measured at the present time by the body weight scale 2. The correction term can be calculated by the following <Expression 2>.

correction term=(current measured body weight−theoretical body weight)×K1 <Expression 2>

In this Expression, the theoretical body weight is an estimated body weight based on the relation between the calorie intake and the calories burned, which is not affected by a weight loss plateau etc. For example, as shown in FIG. 2, when the calories burned is larger than the calorie intake in the weight loss plateau, the theoretical weight is smaller than the current measured body weight.

The theoretical body weight can be calculated by the following <Expression 3>. K1 is an adjustment parameter for determining how much the current measured body weight should be corrected, and for example, it is preferably increased as the difference between the current measured body weight and the theoretical body weight increases.

That is, a value of K1 in the weight loss plateau is preferably larger than that in a smooth period during which weight loss proceeds smoothly. Note that the weight loss plateau is generated due to the body homeostasis of the dieter, the loss of muscle mass due to weight loss, and the hormonal imbalance due to weight loss.

theoretical body weight=previous measured body weight+(calorie intake-calories burned)×K2 <Expression 3>

In this Expression, the previous measured body weight is the body weight of the dieter measured last time by the body weight scale 2. Further, the calorie intake is the calorie intake within a predetermined period (e.g., a period from when the dieter was weighed last time to when the dieter was weighed at the present time). Further, the calories burned are the calories burned within the predetermined period.

At this time, the calorie intake within the predetermined period can be calculated based on food history data. Further, the calories burned within the predetermined period can be calculated based on personal data and exercise history data. Note that the calories burned can be calculated by a general method for calculating calories burned.

K2 is an adjustment parameter for determining how much the value obtained by subtracting the calories burned from the calorie intake is reflected in the previous measured body weight, and can be set based on, for example, the relation among the past measured body weight, the calorie intake, and the calories burned. That is, K2 can be set based on the amount of decrease in the measured body weight relative to the value obtained by subtracting the calories burned from the calorie intake in the past.

As shown in FIG. 2, the difference between the corrected body weight calculated as described above and the measured body weight is small in the smooth period of weight loss, and the difference between the corrected body weight calculated as described above and the measured body weight is large in the weight loss plateau, that is, the value of the corrected weight calculated as described above is a value obtained by smoothing the measured weights of the dieter on a diet. The calculation unit 53 transmits the above corrected body weight data, correction term data, theoretical body weight data, K1 data, and K2 data to the DB 6.

The control unit 54 controls the body weight scale 2 so that the corrected body weight is displayed on the display unit 2b of the body weight scale 2 based on the corrected body weight data. The DB 6 stores personal data, meal history data, exercise history data, corrected body weight data, correction term data, theoretical body weight data, K1 data, K2 data, and the like.

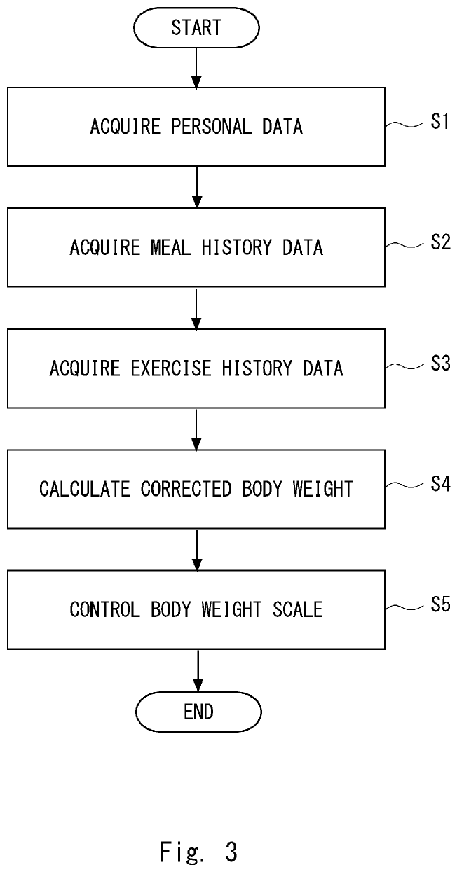

Next, an example of a flow of processes performed by the processing apparatus 5 according to this embodiment will be described. FIG. 3 is a flowchart showing the flow of processes performed by the processing apparatus according to this embodiment. Note that the processes performed by the processing apparatus 5 described below are processes performed when a dieter steps on the body weight scale 2 to measure the dieter's body weight. First, the personal data acquisition unit 51 acquires personal data of a dieter from the DB 6 (S1).

Next, the meal history data acquisition unit 52a of the life data acquisition unit 52 acquires meal history data (S2). Specifically, the meal history data acquisition unit 52a acquires meal data associated with meal time data from the DB 6, estimates a meal menu based on the meal data, and estimates food included in the meal menu and the quantity thereof.

Further, the meal history data acquisition unit 52a calculates the calorie intake of the meal menu based on the food included in the meal menu and the quantity thereof. By doing so, the meal history data acquisition unit 52aacquires the meal history data in which the meal menu, the meal time, and the calorie intake are associated with each other. The meal history data acquisition unit 52a transmits the meal history data to the DB 6.

Next, the exercise history data acquisition unit 52b of the life data acquisition unit 52 acquires exercise history data of the dieter (S3). Specifically, the exercise history data acquisition unit 52b acquires exercise data associated with exercise time data from the DB 6, and estimates an exercise content and the amount of exercise based on the exercise data.

By doing so, the exercise history data acquisition unit 52b acquires the exercise history data in which the exercise content, the exercise time, and the amount of exercise are associated with each other. The exercise history data acquisition unit 52b transmits the exercise history data to the DB 6. Note that the processes of S2 and S3 may be performed in the reverse order.

Next, the calculation unit 53 calculates the corrected body weight based on a correction term generated from the personal data and the life data and a measured body weight of the dieter (S4). That is, the calculation unit 53 calculates the corrected body weight based on the above-described <Expression 1> to <Expression 3>. Then, the calculation unit 53 transmits corrected body weight data and the like to the DB 6.

Next, the control unit 54 controls the body weight scale 2 so that the corrected body weight is displayed on the display unit 2b of the body weight scale 2 based on the corrected body weight data (S5). By doing so, as shown in FIG. 2, the corrected body weight in which the difference between it and the measured body weight is small in the smooth period of weight loss and the difference between it and the measured body weight is large in the weight loss plateau, that is, the corrected body weight having a value obtained by smoothing the measured weights of the dieter on a diet, can be displayed on the display unit 2b of the body weight scale 2.

As described above, the body weight display system 1 and the processing apparatus 5 according to this embodiment calculate the corrected body weight in which the difference between it and the measured body weight is small in the smooth period of weight loss and the difference between it and the measured body weight is large in the weight loss plateau, that is, the corrected body weight having a value obtained by smoothing the measured weights of the dieter on a diet. Therefore, even in the weight loss plateau, the dieter can acquire information indicating that weight loss is smoothly progressing, and thus the motivation of the dieter to lose weight can be prevented from being reduced.

### Second Embodiment

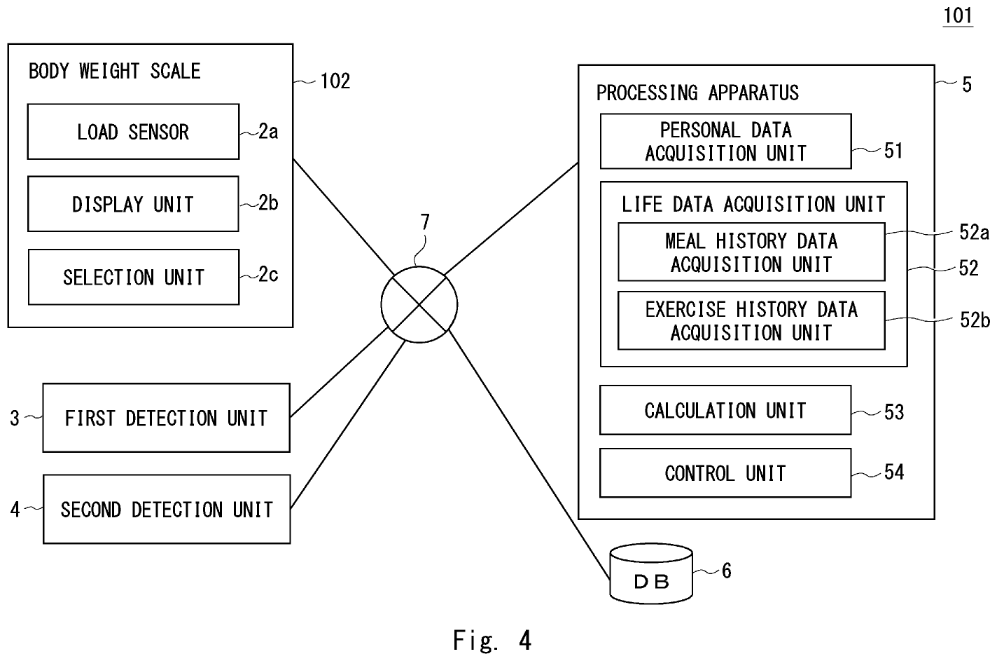

FIG. 4 is a block diagram showing a configuration of a control system of a body weight display system according to this embodiment. As shown in FIG. 4, a configuration of a body weight display system 101 is substantially similar to that of the body weight display system 1 according to the first embodiment, except that a body weight scale 102 includes a selection unit 2c for selecting whether to display a measured body weight or a corrected body weight of a dieter on the display unit 2b.

The selection unit 2c is an input apparatus such as a touch panel, and is operated by a dieter. By this configuration, the dieter can appropriately select whether to display a measured body weight or a corrected body weight of the dieter on the display unit 2b.

### Other Embodiments

For example, in the above first and second embodiments, although the corrected body weight is calculated regardless of whether a dieter is in the smooth period of the weight loss or the weight loss plateau, that is, it is calculated in both the smooth period of the weight loss and the weight loss plateau, the corrected body weight may be calculated only in the weight loss plateau. In this case, when the difference between the theoretical body weight calculated from the personal data and the life data and the measured body weight of the dieter is equal to or greater than a preset threshold, the calculation unit 53 may estimate that the dieter is in the weight loss plateau, and calculate the corrected body weight in the weight loss plateau.

For example, in the above first and second embodiments, although the meal data and the exercise data are acquired by a sensor, they may be acquired by a dieter performing input through an external input unit.

For example, in the above first and second embodiments, although the processing apparatus 5 includes the control unit 54, the body weight scales 2 and 102 may instead include the control unit 54.

For example, some or all of the above-described processes may be performed by a computer program. That is, a control computer comprising the processing apparatus executes the program, thereby performing the control of the above processing apparatus. The above-described program includes instructions (or software codes) that, when loaded into a computer, cause the computer to perform one or more of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

Note that the present disclosure is not limited to the above-described embodiments and may be changed as appropriate without departing from the scope and spirit of the present disclosure.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A processing apparatus comprising:
a personal data acquisition unit configured to acquire personal data including body weight data of a dieter;
a life data acquisition unit configured to acquire life data of the dieter; and
a calculation unit configured to calculate a corrected body weight based on a correction term generated from the personal data and the life data and a measured body weight of the dieter.

2. The processing apparatus according to claim 1, wherein the calculation unit estimates a weight loss plateau of the dieter based on a difference between a theoretical body weight calculated from the personal data and the life data and the measured body weight of the dieter, and calculates the corrected body weight in the weight loss plateau.

3. The processing apparatus according to claim 1, comprising a control unit configured to control a body weight scale so that it displays the corrected body weight.

4. The processing apparatus according to claim 1, wherein the life data acquisition unit acquires exercise history data or meal history data of the dieter as the life data.

5. A body weight display system comprising:
the processing apparatus according to claim 1; and
a body weight scale configured to display the corrected body weight.

6. The body weight display system according to claim 5, comprising a detection unit configured to detect a meal consumed by the dieter.

7. The body weight display system according to claim 5, wherein the body weight scale comprises a selection unit configured to select whether to display the measured body weight or the corrected body weight of the dieter.

8. A processing method comprising:
acquiring personal data including body weight data of a dieter;
acquiring life data of the dieter; and
calculating a corrected body weight based on a correction term generated from the personal data and the life data and a measured body weight of the dieter.

9. A non-transitory computer readable medium storing a processing program for causing a computer to:
acquire personal data including body weight data of a dieter;
acquire life data of the dieter; and
calculate a corrected body weight based on a correction term generated from the personal data and the life data and a measured body weight of the dieter.
